(19) United States
(12) Patent Application Publication
(10) Pub. No.: US 2025/0172460 A1
(43) Pub. Date: May 29, 2025

(54) METHODS AND SYSTEMS FOR EVALUATION OF TISSUE SAMPLES

(71) Applicant: Empiri, Inc., Houston, TX (US)

(72) Inventors: Connor Davis Workman, Chicago, IL (US); Tania Corina Margain Lozano, Austin, TX (US); Kyuson Yun, Houston, TX (US); Thomas Donghyun Gallup, Houston, TX (US); David Forest Gallup, Houston, TX (US)

(21) Appl. No.: 18/729,525

(22) PCT Filed: Jan. 19, 2023
(86) PCT No.: PCT/US2023/011159

§ 371 Date: Jul. 17, 2024

Related U.S. Application Data
(60) Provisional application No. 63/301,038, filed on Jan. 19, 2022.
(60) Provisional application No. 63/439,803, filed on Jan. 18, 2023.

(51) Int. Cl. G01N 1/28; G01N 1/00

(52) U.S. Cl. CPC G01N 1/286; G01N 2001/002; G01N 2001/2873

(57) ABSTRACT

Systems and methods for evaluation of a live tissue sample are provided including a well-insert that may be capable of maintaining an air-liquid interface for three-dimensional (3D) cell cultures. The well-plate insert may include a semi-permeable filter. A tissue sample may be placed within the wells to rest on the top of the filter where it can retrieve nutrients from the media below the filter while still being exposed to air throughout the duration of the cell culture. Filter assemblies may be used to float and support tissue samples placed on top for transfer after slicing. An automated tissue slicer and handler that may cut thin slices of tissue samples and transfer them to a filter assembly. A tissue culture plate may receive slices caught by the filter assembly to be sent for subsequent processing of the slices.

### CROSS-REFERENCE TO RELATED APPLICATIONS

This application is related to U.S. provisional patent application No. 63/301,038, filed Jan. 19, 2022, pending, entitled “Automated Systems and Methods for Processing Biological Samples” and U.S. provisional patent application No. 63/439,803, filed Jan. 18, 2023, entitled “Methods and Systems for Evaluation of Tissue Samples”. Each of these applications is hereby incorporated by reference as though fully set forth herein.

### FIELD OF THE DISCLOSURE

The present disclosure generally relates to evaluation of tissue samples, and more particularly, systems and methods for evaluation of tissue samples.

### BACKGROUND

Mammalian organs/tissues are composed of different cell types that form a 3-dimensional (3D) structure with specific architecture and cell: cell interactions, referred to as ecosystems. Current techniques for ex vivo mammalian tissue cultures involve the destruction and dissociation of the tissue ecosystem to acquire a set of cells used to begin a cell culture in two dimensions (2D). While some techniques exist for 3D cultures, such as organoids, hydrogels, and micro-engineered 3D scaffolds, these techniques are artificial, not representative of naturally occurring animal/human tissue ecosystems, time consuming, and are expensive.

Currently when mammalian tissue such as surgical resections or biopsies are transferred to the laboratory in a manner that will maintain viability for ex vivo culture, the tissues are put in a tube, such as a standard 5 ml, 15 ml or 50 ml tube, which may be filled with a liquid buffer solution and placed on ice or cold packs to keep cold. The buffer solution with the tissue is then removed, and the tissue is picked up using forceps or other manual methods that can stress the tissue, cause it to break apart, or otherwise damage it. Additionally, the motion caused by transport can cause the tissue to disintegrate into smaller pieces making it difficult to find and process. Currently, there are no methods used to minimize damage caused by fluid motion during transport and handling, and no methods to make tissue handling at the laboratory systematic and consistent.

Current methods for slicing live, not fixed or frozen tissue into small slices use vibratomes or tissue choppers. The sample and slicing mechanism occurs in a liquid buffer solution and the released tissue slice floats free into the buffer and is manually picked up from the solution. Current methods do not employ an automated means of handling the live tissue slice after it has been cut. Furthermore, current devices do not allow controlled sterilization of mechanisms that come in contact with samples.

### SUMMARY

Embodiments of the present disclosure may provide a system for processing a live tissue sample comprising: a funnel-shaped structure; and a tube filled with buffer, wherein the funnel-shaped structure is inserted in the tube to transport a tissue sample. The tube may be a 5 ml tube, a 15 ml, or a 50 ml tube. The funnel-shaped structure may be a thin plastic mesh. The structure may be solid; formed of plastic, metal, and/or a gel-like substance; and separable into two parts, and the two parts may be hinged. The funnel-shaped structure may have an opening at a tapered portion of the funnel-shaped structure, wherein buffer flows or is wicked out while the tissue sample remains within the funnel-shaped structure.

Other embodiments of the present disclosure may provide a system for processing a live tissue sample comprising: at least one filter assembly comprising: a filter; and a ring made of material that may be connected to outer portions of the filter, wherein the ring may provide buoyancy for the filter to float on the top surface of a fluid. The filter may be hydrophobic on a side that is not in contact with the ring and hydrophilic on an opposed side that is in contact with the ring. The filter may have a pore size from about 2 μm to about 10 μm. The ring may be formed of biologically inert plastic, a blend of biologically inert waxes, and/or a wrapping film/tape.

Further embodiments of the present disclosure may provide a system for processing a live tissue sample comprising: a well-plate insert, the well-plate insert comprising: at least one column; and a semi-permeable filter attached to a bottom of each column, wherein the semi-permeable filter is capable of maintaining an air-liquid interface for the tissue samples which are placed within the column, wherein when the well-plate insert is placed into a column with liquid at the bottom, the well-plate insert may be capable of displacing the media without flooding an inner chamber of the well plate where the tissue samples sit. The semi-permeable filter may have a pore size from about 2 μm to about 10 μm. Microparticles and/or nutrients within the media may travel upwards past the semi-permeable filter without allowing media to permeate into an air chamber within the well plate.

Embodiments of the present disclosure include a system for processing a live tissue sample, comprising: a sample holder configured to receive a live tissue sample; a bracket configured to receive the sample holder; an indexing mechanism configured to move the live tissue sample in precise increments; and a blade operatively connected to the sample holder that is configured to cut the live tissue sample. In some instances, the indexing mechanism comprises a linear actuator that comprises a plunger configured to move the live tissue sample towards the blade. In some cases, the live tissue sample is moved from about 200 micrometers to about 400 micrometers. The indexing mechanism may comprise a cutting surface configured to receive a controlled amount of fluid sufficient to lubricate the cutting surface and the live tissue sample. The fluid provided may be in the form of a mist having at least one droplet. The system may also comprise a receptacle operatively connected to the sample holder that is configured to receive a section of the live tissue sample after the section is cut by the blade. In some cases, the receptacle is positioned to contact the fluid. In some instances, the embodiments may comprise a user interface, and the system may be configured for automated operation with no more than one single live tissue sample. In this regard, single use operation of the system and any or all of its components may include multiple rounds of use, but refers to the use with a single live tissue sample and the avoidance of any cross-contamination between different samples.

Embodiments of the disclosure may include a method of processing a live tissue sample comprising: providing a well plate comprising a filter assembly that is operatively connected to a blade; cutting the live tissue sample with the blade; and obtaining a section of the live tissue sample. In some cases, embodiment may further comprise contacting the live tissue sample with a fluid configured to generate a droplet of the fluid sufficient to lubricate the live tissue sample; contacting the droplet with the filter assembly; and contacting the section of the live tissue sample with the droplet contacted filter assembly. In some instances, these methods may comprise a user interface, and the system may be configured for automated operation with no more than one single live tissue sample. In this regard, single use operation of the system and any or all of its components may include multiple rounds of use, but refers to the use with a single live tissue sample and the avoidance of any cross-contamination between different samples.

Additional embodiments of the present disclosure may provide an automated tissue slicer and handler (ATSH) comprising: a bracket that receives a sample holder having a tissue sample such as a surgical resection or biopsy embedded in a supporting structure such as agarose; an indexing mechanism for pushing the tissue sample out of the bottom of the sample holder in controlled increments; and a blade positioned below the sample holder to cut thin slices off the bottom of the sample. The indexing mechanism may be a linear actuator that indexes the tissue by moving a plunger in a downward direction. The linear actuator may be capable of pushing controlled amounts of the embedded tissue sample out of the bottom of the sample holder. The linear actuator may be capable of indexing 200-400 micrometer thick slices of the tissue sample. There may be a slicing surface receiving controlled amounts of fluid to lubricate the cutting surface and keep the tissue sample from drying out. Controlled amounts of fluid may be provided by applying a fine mist. The system also may include a receptacle positioned below the sample holder capable of receiving individual slices of the tissue sample as they are sliced off the bottom of the tissue sample. The receptacle may be raised until it touches fluid applied to lubricate the cutting surface and then the receptacle is lowered. A robotic arm may be positioned below the sample holder that is capable of holding a well plate having liquid media in each well, the well plate containing a filter assembly that floats on top of the liquid media in each well that catches slices by touching the filter assembly to the droplet of buffer. A tissue holder, such as a half of a small plastic funnel, having a tip where the sample settles may be inserted inside the sample holder and swirled in the agarose to detach the tissue sample from the tissue holder and embed it in the agarose. The sample holder may have a first portion where the tissue sample has been embedded or encased in agarose and a second portion, which is part of the ATSH, that receives the first portion containing the embedded tissue sample and holds the first portion in place and connected to the ATSH to prepare the tissue sample for slicing. It should be appreciated that the embedding may be done in the first portion in embodiments of the present disclosure such that a tip of the funnel may be inserted and swirled in liquid agarose before the agarose is cooled and solidified. While the sample holder is described as a two-part assembly, there may be other embodiments of the present disclosure where more or fewer than two parts may be provided. The tissue sample may be indexed from a bottom portion of the tissue sample and pushed with a plunger from the top. The ATSH also may include a pump that aspirates slices of the tissue sample as they are sliced from the sample. The ATSH may further include a removable reservoir of buffer that connects to a pump that can dispense the buffer at a controlled continuous flow rate or in controlled, discrete amounts for the purpose of lubricating the agarose as it is pushed out of the sample holder or to place droplets on the exposed, bottom portion of the sample to keep the sample moist, keep the cutting surface clean, or lubricate the slicing operation; and a suction pump that collects the slices. It should be appreciated that the components that are exposed to the tissue sample may also be single use, disposable components facilitating easy, aseptic processing of samples by removing the single use components that were used on the previous sample and replacing them with new components for a new sample. These single use components can include but would not be limited to the agarose, sample holder, cutting blade, filter assembly and well plates, sample pusher that pushes on the top of the sample, and lubricating fluid.

Other embodiments of the present disclosure may provide a method to transfer a slice of a tissue sample onto a filter assembly comprising: holding a tissue sample above a slicing blade positioned above a well plate containing a filter assembly; and using the slicing blade, cutting a slice from a bottom portion of the tissue sample. The method also may include dispensing lubricating fluid onto an exposed portion of the tissue sample to generate a droplet of the lubricating fluid to lubricate the tissue sample; raising the filter assembly to touch the droplet, wherein surface tension causes the droplet to pull into the filter assembly; and after cutting the slice, lowering the slice to rest on the filter assembly.

### BRIEF DESCRIPTION OF THE DRAWINGS

Reference is made to the following description, taken in conjunction with the accompanying drawings, in which:

FIG. 1 depicts a top isometric view of well-plate insert according to an embodiment of the present disclosure;

FIG. 2 depicts a bottom isometric view of a well-plate insert according to an embodiment of the present disclosure;

FIG. 3 depicts a filter assembly and filter according to an embodiment of the present disclosure;

FIG. 4 depicts a transfer process to transfer a slice onto a filter assembly according to an embodiment of the present disclosure;

FIG. 5 depicts a self-standing, automated instrument in which the automated tissue slicer and handler is one component, that performs treatment sensitivity assays according to an embodiment of the present disclosure;

FIGS. 6A-6B depict top and side views of a filter assembly with a supporting structure below the filter according to an embodiment of the present disclosure;

FIGS. 6C-6E depict top and side views of a filter assembly using air pockets according to an embodiment of the present disclosure;

FIGS. 6F-6G depict side and top views of an integrated well plate and filter assembly according to an embodiment of the present disclosure;

FIGS. 6H-6K depict top and side views of a floating filter assembly and well plate according to an embodiment of the present disclosure;

FIG. 7 depicts a Sample Transfer Cassette (STC) and placement of a tissue sample into an STC and transfer into agarose according to an embodiment of the present disclosure;

FIGS. 8A-8B depict slicing and plating methodologies according to embodiments of the present disclosure;

FIG. 8C depicts a method for tissue slicing according to an embodiment of the present disclosure;

FIG. 9 depicts a method for tissue slicing and placing according to an embodiment of the present disclosure;

FIG. 10 depicts a workflow from STC to placing inside a tissue culture plate according to an embodiment of the present disclosure;

FIG. 11 depicts an ATSH according to an embodiment of the present disclosure; and

FIGS. 12A-12C depict system architecture of handling processing after tissue slicing and handling has been completed in embodiments of the present disclosure.

### DETAILED DESCRIPTION

Embodiments of the present disclosure may provide automated systems and methods for processing biological samples, particularly live tissue. As described herein, “tissue” may be defined as a physically attached collective of cells where physical attachment may include association in a extracellular matrix as well as cell-cell adhesion via occluding junctions, adhering junctions, desmosomes, anchoring junctions and gap junctions. The tissue as used herein may include normal, pre-cancerous and cancerous cells. Further, “live” may be defined as metabolically active or viable where living versus dead cells may be distinguished via cell membrane permeable indicators and/or dyes. “Live tissue” as used herein is defined to be tissue that contains live cells, although a live tissue sample may also include dead cells. Also, “single-use” may be defined to include one-time usage, disposable, not reusable and/or non-renewable. This may include conditions associated with the avoidance of cross sample- contamination; any pre-use or post-use sterilization, decontamination and/or disinfection, or the minimization of any harmful or deleterious effect as defined by legal or regulatory standards. In this regard, single use may include multiple uses of a system or repeated method steps with a single live tissue sample.

In an embodiment of the present disclosure, a well-insert may be provided that may be capable of maintaining an air-liquid interface for three-dimensional (3D) cell cultures, specifically harvested mammalian tissue independent of external factors including, but not limited to the volume of media or evaporation. The well-plate insert according to embodiments of the present disclosure may include a 3-micron filter that may be semi-permeable in that it may allow microparticles/nutrients within the media to travel upwards past the filter without allowing liquid to permeate into the air chamber within the well plate. While the filter is described as 3-micron, it should be appreciated that there may be embodiments of the present disclosure where the filter pore size is larger or smaller. The filter and surrounding walls of the insert may independently not allow liquid into the air chamber. A tissue sample may be placed within the wells to rest on the top of the filters sitting at the bottom of the well plate where it can retrieve nutrients from the media below the filter while still being in contact with air above the tissue throughout the duration of the tissue culture. In an embodiment of the present disclosure, the tissue sample may be 400-microns in thickness; however, it should be appreciated that the thickness may be larger or smaller in some embodiments of the present disclosure.

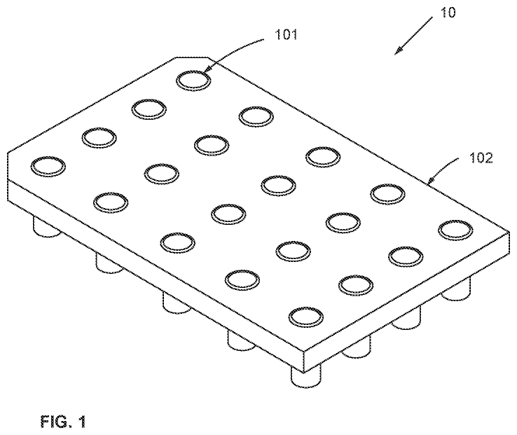

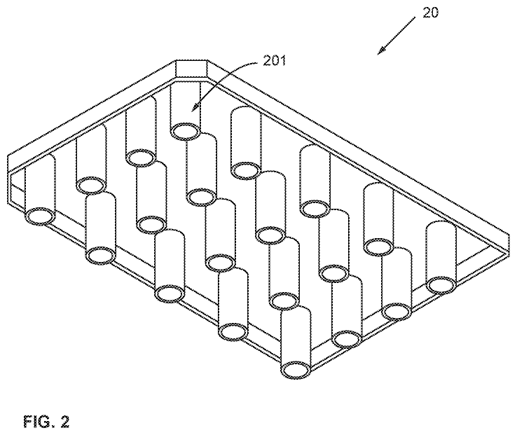

FIG. 1 depicts a top isometric view of well-plate insert 10 according to an embodiment of the present disclosure. More specifically, FIG. 1 shows the form factor of the inserts to be oriented for a 24 well plate with a lid-like perimeter. While insert 10 is depicted for a 24 well plate, it should be appreciated that more or fewer wells may be provided without departing from the present disclosure. Holes 101 visible on top of lid 102 may extend through lid 102, and the semi-permeable filter is capable of being attached to the bottom of each column (see FIG. 2).

FIG. 2 depicts a bottom isometric view of well-plate insert 20 according to an embodiment of the present disclosure. As depicted herein, the bottom of columns 201 may have a semi-permeable filter securely attached so that the tissue can sit on top of the filter. When insert 20 is placed into a well-plate with media, it can displace the media without flooding the inner chamber where the tissue sits.

The dimensions of the well plate may be adapted to a 96, 48, 24, 12, and 6 well plate in embodiments of the present disclosure. However, other dimensions, configurations, and numbers of wells may be provided without departing from the present disclosure. It should be appreciated that the walls of the well plate may not have any openings, such as holes or pores, to allow liquid to enter from sides of the chamber but can include holes/pores near the top that are above the liquid. The inner diameter and outer diameter of each well-insert column can be varied as well as the heights of each column in embodiments of the present disclosure. It also should be appreciated that the filter pore size can be varied to allow different particles to pass through in embodiments of the present disclosure.

Filter assemblies may be used in embodiments of the present disclosure. Filter assemblies may have 3 μm pore size filters that are hydrophobic on one side and hydrophilic on the other side. When placed with the hydrophobic side down onto a media/buffer solution, the filter assembly may float and support the biopsy which may be placed on top. The 3 μm pore size may ensure that T-cells do not pass through the filter. However, other sizes of filters may be used depending on the type of tissue and test objectives without departing from the present disclosure. It also should be appreciated that there may be embodiments of the present disclosure where the filter itself does not need to be buoyant (i.e., may not be hydrophobic on one side), and other methods of providing buoyancy may be utilized.

The perimeter of the circular cut filter assembly may include a ring of Parafilm wrapping film or other comparable non-toxic, buoyant film which has been attached to the filter assembly. While the filter assembly is described as circular cut, other shapes may be used without departing from the present disclosure. The wrapping film/tape may provide additional buoyancy (while being non-toxic) so that even as the filter assembly is handled, the filter may stay floating on top. A filter according to embodiments of the present disclosure may be a 5 mm circle having a ring of plastic, such as polyethylene terephthalate (PET), that may be approximately 0.5 mm thick and 1 mm in height. This ring may be attached using a non-toxic glue that may be heated and bonded to the filter. However, another non-toxic mechanism for attaching the ring to the filter may be used without departing from the present disclosure. The shape and positioning of the filter may allow the biopsy to be exposed to air on the top surface and exposed to nutrients, coming through the filter, on the bottom surface.

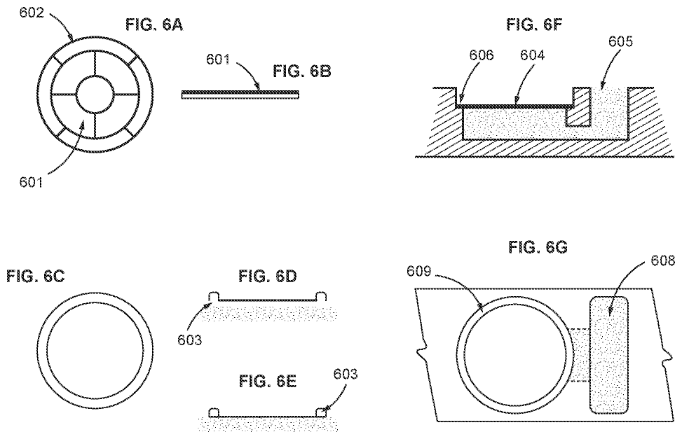

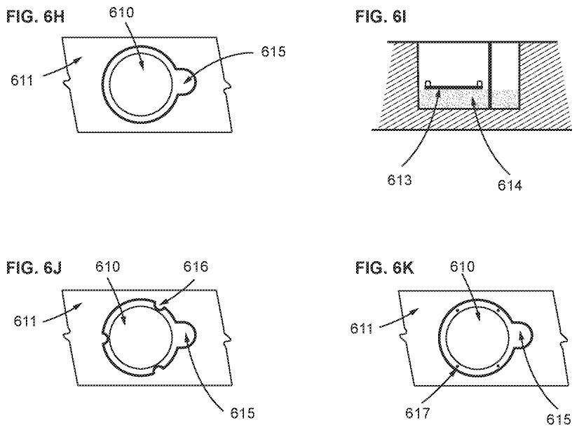

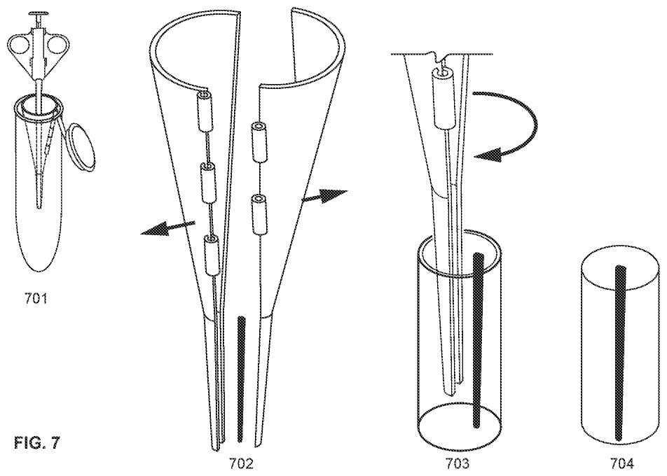

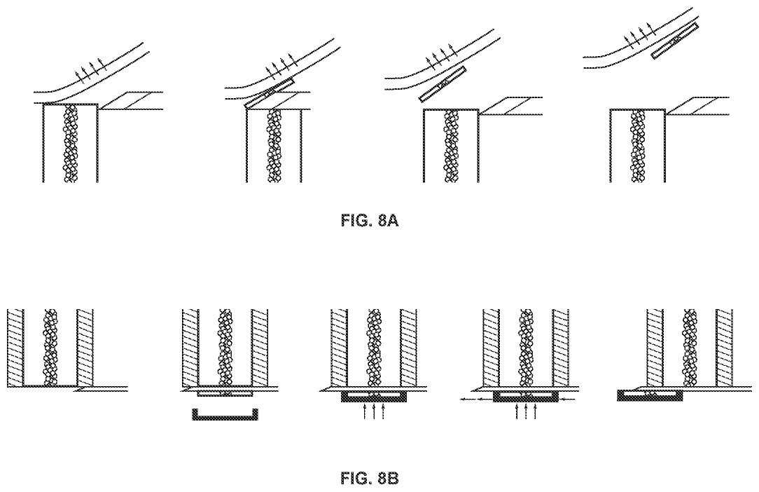

A filter assembly according to embodiments of the present disclosure may maintain an air/liquid interface, including during handling and/or processing of the tissue. The filter assembly may enable easier transfer of a tissue slice from an automated slicer into the filter. As described herein, in embodiments of the present disclosure, the filter assembly may float on top supported by the filter itself being buoyant such as depicted in FIGS. 6C-6E. More specifically, FIG. 6C depicts a top view of a filter assembly being buoyant. The filter may be hydrophobic on one side, or the filter may be less dense than the liquid on which the filter may float. This filter may include sealed air pockets and may be open to the liquid below the filter. FIG. 6D depicts a side view with open air pockets 603 exposed to liquid, and FIG. 6E depicts a side view with a sealed air pocket where the filter shape may provide for sealed air pockets 603. Alternatively, the filter may be supported by a separate supporting structure that is buoyant.

FIGS. 6A-6B depict top and side views of a filter assembly with a supporting structure below the filter according to an embodiment of the present disclosure. Filter 601 may be generally supported from the bottom and may be held via a fastening mechanism including, but not limited to, adhesive, heat bonding, chemical bonding, clamping, and poking through. Outer ring 602 may be provided on the bottom of the filter that may be held via one of the fastening mechanisms. In another alternative, an article, such as an outer ring, may be provided on top of the filter and held via one of the fastening mechanisms. FIG. 6A depicts a bottom view of the filter assembly showing filter bottom 601 with plastic or other buoyant supporting structure 602 on the bottom, and FIG. 6B depicts a side view showing filter 601 on top of the supporting structure.

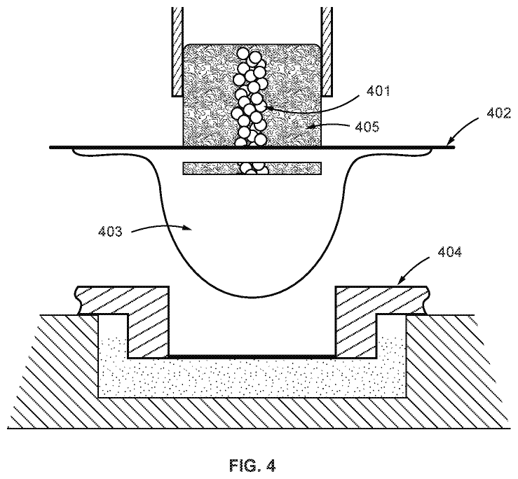

In further embodiments of the present disclosure, a fixed filter may rest on top of the well plate assembly as shown in FIG. 4. These fixed filter assemblies can be assembled in an array such as depicted in FIGS. 1 and 2.

In additional embodiments of the present disclosure, the filter may be built into a well plate assembly, such as depicted in an integrated well plate and filter assembly of FIGS. 6F-6G. As depicted herein, filter 604 may be bonded to well plate 606, and the top of buffer solution 605 may be open to air, while the filter may prevent buffer solution to escape to the top surface of the filter, such as in the side view depicted in FIG. 6F. This may enable supply of buffer solution to the area underneath the filter, such as a side “water tower” where gravity may feed the area under the filter with buffer solution. The water tower may allow filling with enough buffer solution such that, even with evaporation, the fluid level may be sufficient to maintain contact with the filter throughout the process. This may be a pressurized bag or other mechanism in embodiments of the present disclosure. FIG. 6G depicts a top view showing a supply of buffer solution 608 wherein filter 609 covers the top of the buffer solution in the area that it covers.

FIGS. 6H-6K depict top and side views of a floating filter assembly and well plate according to an embodiment of the present disclosure. More specifically, FIG. 6H depicts a top view of a two-part assembly wherein filter assembly 610 is floating on top of fluid at the bottom of a well in a well plate, and the filter assembly may include a pocket or area to allow liquid handling to be done, such as with a pipette or other liquid handling device, without hitting the filter. FIG. 6I depicts a side view of the two-part assembly showing filter 613 relative to buffer/media 614. FIG. 6J depicts a top view of an alternative design of a two-part assembly. Again, filter assembly 610 is depicted floating on fluid at the bottom of a well in a well plate, and filter assembly 610 may include space 615 for pipetting without hitting the filter assembly. The filter assembly may include one or more nubs or protrusions 616 in the side wall of the well plate that may limit the contact area with filter assembly 610 to reduce friction sliding up and down the wall. FIG. 6K depicts a top view of another alternative design of a two-part assembly. Again, the filter assembly 610 is depicted floating on top of fluid at the bottom of a well in a well plate, and filter assembly 610 may include space 615 for pipetting without hitting the filter assembly. The filter assembly may include one or more posts 617 in the well to limit the contact area with the filter assembly to reduce friction sliding up and down the wall. In each of the embodiments of FIGS. 6H-6K, the well plate may protect the floating filter assembly, such as a filter with an outer buoyant ring, from a pipet tip or other device that may need to enter the well plate to perform liquid handling operations, such as adding or removing media reagents. This can be done by adding a recess or alcove to the side of the area where the filter assembly resides. This recess or alcove may be where the pipet or other device enters to perform liquid handling. Within the area where the filter assembly resides, there may be different embodiments. FIGS. 6J and 6K depict configurations wherein the contact points that the walls of the well plate touch the filter assembly may be well-defined. This can minimize friction and result in a more reliable rising and falling of the filter assembly as liquids are added and removed while the filter floats on top.

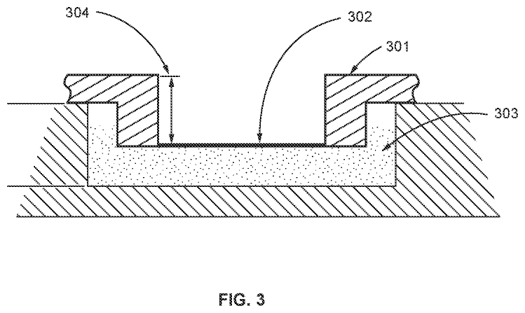

FIG. 3 depicts filter assembly 304 and filter 302 according to an embodiment of the present disclosure. Filter assembly 304 may have an inner wall height of approximately 2.5 mm for a media/buffer solution volume of 200 μl in an 11 mm diameter well, the dimension of a typical 48 well plate. The walls may help filter assembly 304 float even as it is handled and/or bumped. The walls also may provide a rigid surface to hold to manipulate filter assembly 304 in embodiments of the present disclosure. The wall height may facilitate interaction with the droplet on the bottom of the sample to keep the sample from drying out while also lubricating the slicing operation. This wall height may contact the droplet which may center the droplet and make controlled transfer of the biopsy slice to the filter assembly easier. However, it should be appreciated that the wall height of the filter assembly may be reduced in embodiments of the present disclosure to allow easier placement of the slice.

As depicted herein, filter assembly 304 does not float; it rests upon the top of the well plate and the depth of the filter assembly is enough to assure that filter 302 is in contact with media 303 at the bottom of the well and filter 302 may be pushed into the surface of media 303. If filter assembly 304 pushes filter 302 down slightly (˜0.5 mm) into the surface of media 303, then the water pressure may be low enough that media 303 will not come rushing through or “bleed” through filter 302. Filter 302 may stay in contact with media 303, and media 303 will not evaporate enough that filter 302 would lose contact with media 303. Accordingly, through use of filter assembly 304 in embodiments of the present disclosure, evaporation may be reduced as the surface area of the media-to-air interface is reduced, which may reduce variability in media volume due to evaporation or allow for lower volumes of liquid reagents to be used thereby reducing cost and analytical variation.

Minimizing the surface area of filter 302 and the exposed top of media 303 may result in evaporation that can be small enough that only about 0.2 mm of media height may be lost in 3 days of incubation at 37° C. More specifically, FIG. 3 depicts 3-days evaporation of approximately 13 μl, which may be approximately 0.2 mm media height lost in an embodiment of the present disclosure.

While filter 302 and filter assembly 304 are depicted in FIG. 3, it should be appreciated that there may be embodiments of the present disclosure where a filter that is fixed in position may be used, such that a filter assembly which floats on the surface of media 303 may not be necessary. In other embodiments of the present disclosure, an array or rack of wells may be used that are connected and do not “float.” This rack may facilitate lifting more than one filter assembly with filters having biopsies on top at the same time to allow easier liquid handling in downstream operations. This rack of filter assemblies connected together may be shallow (approximately 2.5 mm in height) and may be used like the individual filter assemblies described herein so that the wall height may allow interaction with the droplet to help center and promote controlled transfer of the slice.

FIG. 4 depicts a transfer process to transfer a slice onto filter assembly 404 according to an embodiment of the present disclosure. As depicted herein, sample 401 may be held above slicing blade 402 and then pushed down/indexed from above. Lubricating fluid may be dispensed onto the bottom/exposed portion of sample 401 to lubricate the slice, keep the exposed portion of the sample moist, or keep the slicing blade clean. When filter assembly 404 is raised and it touches droplet 403, surface tension may cause droplet 403 to pull into filter assembly 404. The slice may settle and then it may be lowered so that it may be on filter assembly 404. As depicted herein, a slice may be cut off the bottom of sample 401. The well plate containing filter assembly 404 may be raised until filter assembly 404 touches droplet 403. Filter assembly 404 may then be lowered.

Agarose is a gelatin like substance that melts and solidifies at a certain temperature. Embodiments of the present disclosure use “low melt” agarose 405 which melts at 40° C. This may allow it to be heated up to melting point, tissue such as from a biopsy 401 be embedded in it, and then cooled down to solidify and hold the biopsy in place. The 40° C. melting point means that when the sample is incubated at 37° C. (body temperature), the agarose will be solid, and it can also be melted to permit embedding the biopsy at a temperature that is still low enough that the biopsy will survive and not change its behavior/response from it being too hot.

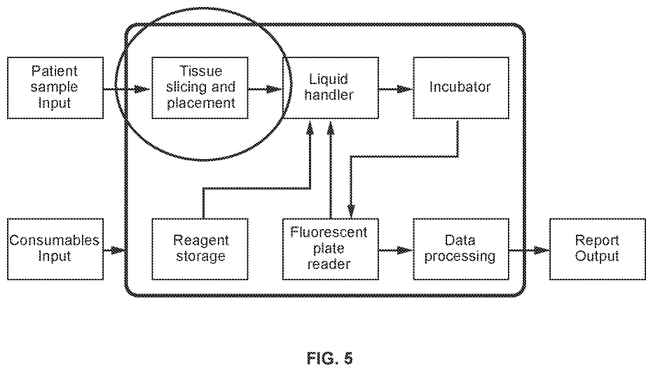

FIG. 5 depicts a self-standing, automated instrument that performs treatment sensitivity assays that include steps performed according to an embodiment of the present disclosure. In embodiments of the present disclosure, the portion of the instrument that performs the initial processing of the tissue be referred to as an automated tissue slicer and handler (ATSH). The portion of the instrument that processes the tissue after the ATSH may be used for liquid handling, plate reading, incubating, and/or reagent management for the downstream processing of the assay.

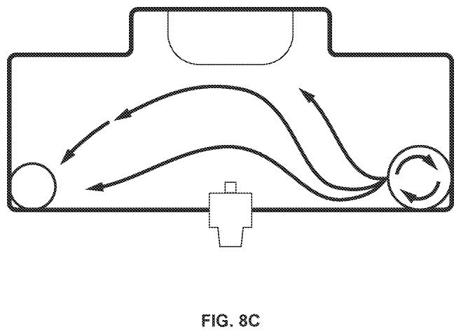

FIG. 7 depicts a hinged design of a sample transfer cassette (STC), a device that accepts input of a tissue sample at the clinic after the tissue sample has been taken, facilitates keeping the tissue more intact during transfer to a facility that will be processing the tissue sample, and makes removal and handling of the sample at said processing facilities easier and less stressful on the tissue. This processing at the facility may be the transfer of the tissue from the STC into agarose. In panel 701, a tissue sample, a biopsy as shown here, may be transferred into a 15 ml tube holding a buffer solution, which may be referred to as the STC in an embodiment of the present disclosure. This transfer process may be performed at a clinic. Once the tissue sample has been placed in the STC, it may be transferred from the clinic to the laboratory.

Panel 702 depicts a hinged, funnel design of a STC according to an embodiment of the present disclosure. The STC may be used to help transfer the sample from the clinic to the laboratory. The STC may fit inside a vessel, such as a standard 5 ml, 15 ml or 50 ml tube, that contains a liquid in which to transport the tissue, such as buffer, media, or PBS. The tissue may be placed into the liquid, and it may settle into the bottom of the removable tissue holding piece. For biopsy needles, they can be swirled in the solution to rinse the buffer off the needle and into the solution. The tissue holding piece may be formed of two parts. They can be hinged, snapped together, and/or held together by a third part such as the cap fitting or walls of the transporting vessel in embodiments of the present disclosure. Once the sample is ready to be used, the tissue holding piece may be pulled out of the transport vessel or tube. The holding piece then can be pulled apart leaving the tissue in one part of the holding piece. At this point, the tissue can be processed. In one embodiment, the part of the holding piece containing the biopsy sample may be flipped on top of a melted agarose bed or twirled inside of a chamber containing agarose or other embedding material. The holding piece can be multiple shapes. In an embodiment. it may be funnel shaped, tapering towards the bottom; however, it could be a straight cylinder, triangular, rectangular, or other shapes in cross section in other embodiments of the present disclosure. It can have a mesh, filter, small hole, or porous material at the bottom so that when lifted out of the transporting vessel, the liquid solution may flow out or could be wicked out by placing an absorbent material on the other side. The tissue would still be held by the holding piece.

Panel 703 depicts transfer of the biopsy from the STC to agarose in an embodiment of the present disclosure. Panel 704 depicts the biopsy embedded in agarose in an embodiment of the present disclosure.

Generally, when a biopsy is taken, the technician puts the needle into buffer solution that is in a 5 ml, 15 ml or 50 ml tube and swirls it around to wash the biopsy off the biopsy needle. Embodiments of the present disclosure may fit inside these tubes and leave room for the technician to swirl the biopsy needle in the buffer solution and then rely on gravity to have the biopsy settle into the funnel/mesh. There are instances, such as with fatty tissue, where the biopsy may float; but, in those situations, when the mesh/funnel is pulled out, the liquid may drain through the bottom, pulling the tissue into the funnel for downstream handling. The STC may fit inside a 15 ml tube that may be constructed from polypropylene (PP) plastic where the biopsy needle may be inserted to release the biopsy into a buffer solution. The biopsy may settle at the tip of the hinged funnel piece. Once the sample is ready to be embedded, the hinged funnel may be pulled out of the 15 ml tube, and excess liquid may be wicked out. The funnel may be hinged so that the biopsy can be released and settled on one half of the funnel (FIG. 7, panel 702). The half containing the biopsy sample may be inserted inside a 5 mm sample holder containing melted agarose (FIG. 7, panel 703). The tip of the STC, containing the tissue sample, may be swirled inside the melted agarose to allow the tissue to detach from the plastic funnel and embed in the agarose. After the tissue sample is in the agarose, the sample holder may be cooled to solidify the agarose, resulting in a fully embedded tissue sample or biopsy (FIG. 7, panel 704). It should be understood that tissue samples besides biopsies, such as surgical resections, may be shipped in the STC, and receive many of the same benefits of less disruptive transport and easier handling and processing in the lab, or processing facility.

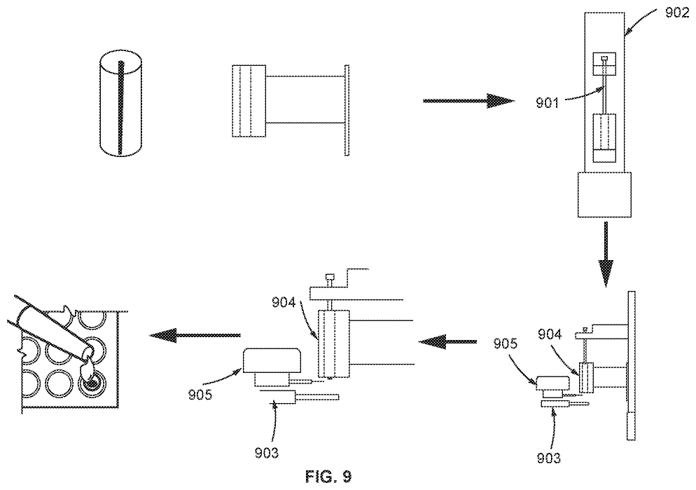

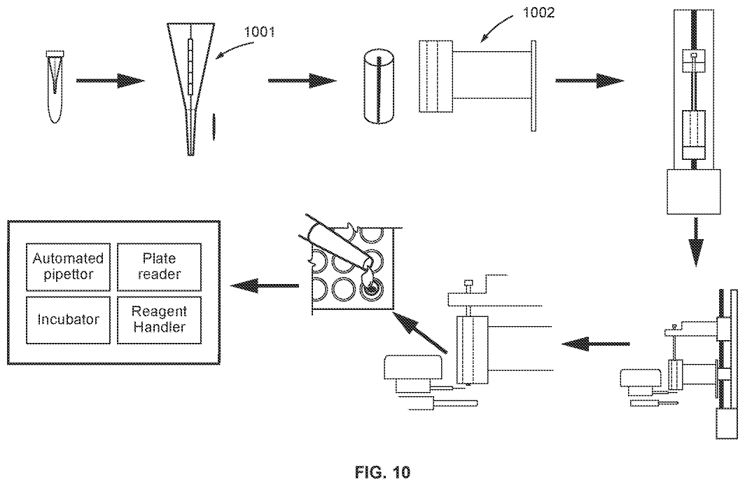

FIGS. 8A-8B depict slicing and plating methodologies according to embodiments of the present disclosure. In an embodiment of the present disclosure, the slicing setup may be submerged in a bath so that the tissue may be sliced from the top down. In another embodiment of the present disclosure, tissue may be sliced from the bottom and pushed with a plunger, thereby making a buffer bath impractical. Rather, dropwise lubrication may be used. In FIG. 8A, capturing and transporting may be done with a pump that may gently aspirate slices as they are coming off the slicer to be picked and placed in a well plate. In this embodiment, slicing may be done from the top down or bottom up. In FIG. 8B, tissue may be captured by lifting an insert or entrapping vessel to trap the sample and gently capture it in the inner portion of the insert. In this embodiment, the slicing may be done from the bottom-up.

FIG. 8C depicts a method for tissue slicing according to an embodiment of the present disclosure. The tissue slice may be captured by generating a flowing current of buffer that may guide the tissue slice to a channel, filter, or collection vessel where it may be collected with a suction pump or mechanical entrapment.

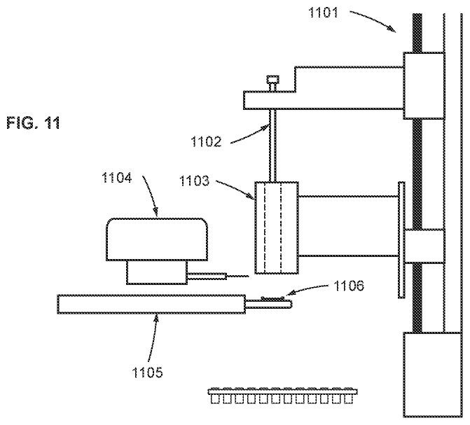

FIG. 9 depicts a method for tissue slicing and placing according to an embodiment of the present disclosure. The tissue sample may be released from the STC and embedded in agarose inside the smooth inner lining of the sample holder. Then the embedded core may be placed inside the sample holder that may be clamped on to the ATSH. The sample holder may be mounted onto bracket 902 where a linear actuator will index the sample in 200-400 micron increments with disposable plunger 901. This assembly can be positioned to allow the vibrating blade to move forward and cut the tissue slices. Robotic arm 903 holding the filter assembly/well plate may be positioned below blade assembly 905 and sample holder 904. A buffer solution may be dispensed dropwise to facilitate capturing the slice with surface tension, lubricating the slice, and keeping exposed tissue moist. The filter assembly may be included inside a modified tissue-culture well.

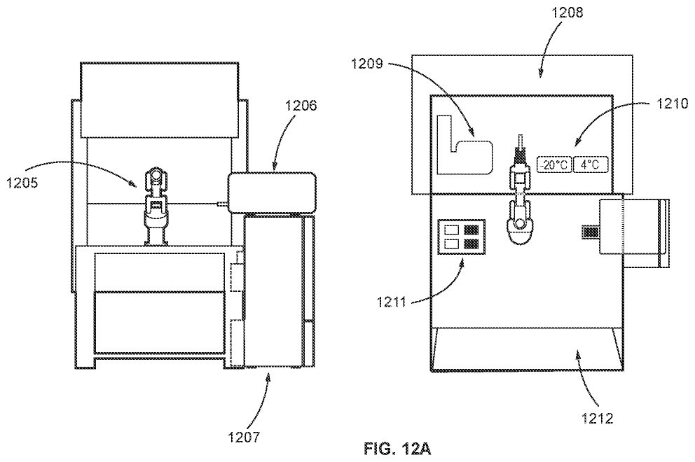

FIG. 10 depicts a workflow from STC to placing inside a tissue culture plate according to an embodiment of the present disclosure. The tissue sample may be deployed into a 15 ml tube containing the STC. STC 1001 may have a hinged design that may keep the tissue sample intact and align the small pieces toward the tip. The tip of the STC may be inserted into an agarose-filled core and swirled to release and embed the sample. The embedded sample may be placed inside the second part of sample holder 1102 to prepare for slicing. The sample holder may be mounted onto a bracket where a linear actuator may index the sample in 200-400 micron increments with a disposable plunger. This assembly can be positioned to allow the vibrating blade to move forward and cut the tissue slices. A robotic arm holding a filter assembly may be positioned below the blade and the sample holder. A buffer solution may be dispensed dropwise to facilitate capturing the slice with surface tension. After each slice is caught, it may be placed inside a well (96-well plate). Subsequently, the plate may be run through the liquid handling system and other processing steps.

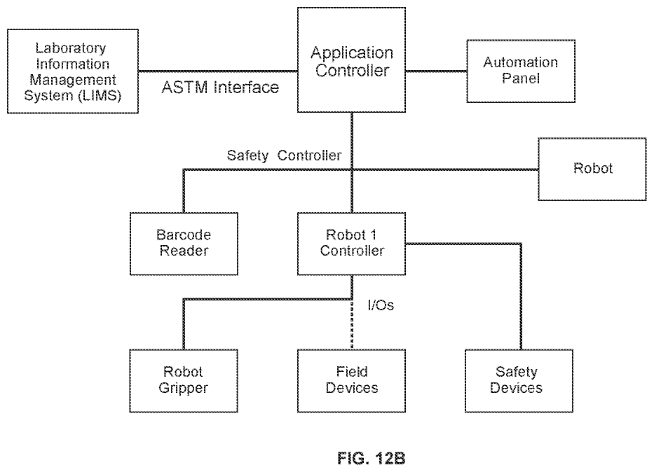

FIG. 11 depicts an ATSH according to an embodiment of the present disclosure. Linear actuator 1101 may be used to index 200-400 micron thick slices by moving plunger 1102 down with 0.003125 mm resolution. Robotic arm 1105 may hold filter assembly 1106 used to catch slices as they are cut using blade assembly 1104 from sample holder 1103. The ATSH may include a tissue culture plate where filter assemblies 1106 containing tissue slices may be inserted. The wells in the tissue culture plate may be prefilled with media. The well plate may be used to hold slices that are held in filter assemblies and sent for subsequent processes of liquid handling and incubation cycles. While not shown, an infusion, syringe, or other type of pump may be dispensing drop-wise amounts of buffer on to the slicing surface to keep the exposed portion of the sample moist and to lubricate the slicing process. This also may serve to help capture slices in a drop of buffer into a filter assembly.

The automated tissue handling and slicing described in these embodiments may eliminate steps that must be performed when doing the process manually. Automated means of handling the sample after it is sliced may be provided. As described herein, this may eliminate steps that must be performed when doing the process manually. Further, as described herein, single use, disposable elements may be used in automated methods according to embodiments of the present disclosure. These single use, disposable elements may aid in avoiding cross contamination from one sample to the next, make sample processing easier, eliminating what is currently a time-consuming process of taking the slicing assembly apart and wiping everything down with alcohol, and facilitate sales of the overall instrument or the automatic tissue slicer and handler at a lower price while selling the single use components either as a package (all the single use components needed to process a single sample) or individually at a price that generates an ongoing profitable revenue stream.

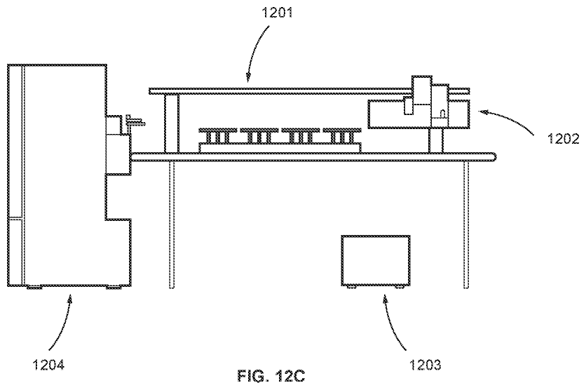

FIGS. 12A-12C depict system architecture of handling processing after tissue slicing and handling has been completed in embodiments of the present disclosure. FIG. 12A depicts system architecture to handle processing after the tissue slicing and handling is complete including robot 1205, fluorescence imager 1206, cell culture incubator 1207, BSC Class A2/B2 1208, tissue slicer 1209, cold blocks 1210, I/O 1211, and/or HEPA laminar flow 1212. FIG. 12B depicts a configuration where there is a central robot that moves the sample between different components of the automation. As depicted herein, one or more robot controllers may be utilized in conjunction with one or more application controllers communicating with an automation panel and/or an information management system over one or more interfaces to operate a robot, components of a robot, such as a robot gripper, field devices, barcode readers, and/or safety devices. FIG. 12C depicts a linear configuration where the sample is moved back and forth primarily linearly between different components of the system including incubator 1201, automated pipettor 1202, plate reader 1203, and/or reagent handler 1204.

In the embodiments described in FIGS. 9 and 10, it should be appreciated that the sample may be transferred from the slicing operation to a tissue holding vessel through use of gravity. The slicing operation would not be lubricated and the slice would fall off on its own or be knocked off with a slight tap or agitation. While lubrication benefits may not be provided in this embodiment, the slice may still be transferred in a controlled fashion.

In an alternative embodiment, liquid may be added so that it may drip down into a holding vessel. While the liquid may need to be eliminated from the excess fluid used from this transfer mechanism, the slice still may be transferred in a controlled fashion. A vacuum may be used to suck up the sample on to a surface which could then transport the slice to another area and then use positive pressure to release the slice and push it via this positive pressure into a holding vessel. The transport could be done a variety of ways including, but not limited to, through use of a robotic arm and/or moving conveyor belt. The robotic arm may include a vacuum at the end, and the moving conveyor belt may include a center section that pulls a vacuum as it moves.

In some embodiments where the slices are being cut from the top of the sample, and the sample, including the cutting surface, is submerged in the media, a tray may be provided on the far side of the sample, with mesh walls and a mesh bottom, such that when the slice comes free it is pushed into the mesh area by a feature of the cutting or slicing operation. The blade could then tilt and lift out of the media leaving the slice in the mesh (like a net with some rigidity) and then the net could be lifted out in an automated fashion.

There may be other embodiments wherein the slice is pushed into a known area which is then raised out of the media bath and can be picked up by vacuum, or the lifting surface is movable and deposits the slice elsewhere. In some embodiments, the sample may be laid on its side and sliced vertically with slices coming off the end of the sample. As they come off, the slices may fall onto a surface, in a domino-like fashion, that can then be used to transport the slice to a new location or the surface that the slice falls onto could be the filter assembly itself. The surface could be porous to let the lubricating liquid pass through or be wicked through leaving primarily the tissue slice on top.

In other embodiments, a shallow cap may be provided that is slightly deeper than the slice thickness. The cap may be positioned on the bottom of the exposed sample that is to be sliced off, leaving a gap that is large enough to permit the blade to go between it and the sample. Thus, when the blade has finished the slicing, the slice may be inside the cap which could be moved sideways, or lowered to transport the slice for later processing. The cap configuration could be a filter assembly with sides large enough to capture the slice. The cap could come up containing liquid inside it to eliminate the need to add fluid to the bottom of the slice during the slicing operation or it could have holes or gaps in the walls that are large enough to allow fluid to flow into the cutting area but small enough to prevent the slice some coming out.

Cutting can be done by a blade that oscillates back and forth in an X direction and then moves forward in a Y direction that is perpendicular to the X direction. The X-Y plane may be oriented substantially perpendicular to the sample. A rotating blade could be used in some embodiments of the present disclosure. The blade could be made out of one or more materials including, but not limited to, steel, other metals, obsidian (volcanic glass), or other similar materials.

Some embodiments of the present disclosure may use the flowing current of media as described herein. A net at the end, that the sample flows into, may be on the end of a robot or part of a movable system and can then be used to transport the sample.

Further embodiments of the present disclosure may include a vision system that may direct a net, or other restraining structure, to pick up the floating slice after it has come free from the top of the sample when the slicing is done in a similar configuration to how it is done manually now, slicing from the top and the sample immersed in media. The agarose could be given a die or coloration to make it easier to see when floating in the media.

Other embodiments may include a small cap over the top of the sample that may be used while slicing the sample from the top down with the sample immersed in media. The cap may have a slight suction and may be positioned so that the blade can go under it. The suction may pull the sample into the cap as it is sliced to be removed and transferred to another location upon completion of the slicing step.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A system for processing a live tissue sample, comprising:
a sample holder configured to receive a live tissue sample;
a bracket configured to receive the sample holder;
an indexing mechanism configured to move the live tissue sample in precise increments; and
a blade operatively connected to the sample holder that is configured to cut the live tissue sample.

2. The system of claim 1, wherein the indexing mechanism comprises a linear actuator that comprises a plunger configured to move the live tissue sample towards the blade.

3. The system of claim 2, wherein the live tissue sample is moved from about 200 micrometers to about 400 micrometers.

4. The system of claim 1, further comprises a cutting surface configured to receive a controlled amount of fluid sufficient to lubricate the cutting surface and the live tissue sample, wherein the indexing mechanism is operatively connected to the cutting surface.

5. The system of claim 4, wherein the fluid is provided in the form of a mist having at least one droplet.

6. The system of claim 4, further comprising:
a receptacle operatively connected to the sample holder that is configured to receive a section of the live tissue sample after the section is cut by the blade.

7. The system of claim 6, wherein the receptacle is positioned to contact the fluid.

8. The system of claim 1 further comprising a user interface and wherein the device is configured for automated operation with no more than one single live tissue sample.

9. A method of processing a live tissue sample comprising:
providing a well plate comprising a filter assembly that is operatively connected to a blade;
cutting the live tissue sample with the blade; and
obtaining a section of the live tissue sample.

10. The method of claim 9, further comprising:
contacting the live tissue sample with a fluid configured to generate a droplet of the fluid sufficient to lubricate the live tissue sample;
contacting the droplet with the filter assembly; and
contacting the section of the live tissue sample with the droplet contacted filter assembly.

11. The method of claim 9, further comprising providing a user interface operatively connected to a device configured to perform the steps of the method in an automated operation with no more than one single live tissue sample.

12. A system for processing a live tissue sample comprising:
a funnel-shaped structure; and
a tube filled with buffer,
wherein the funnel-shaped structure is configured for insertion into the tube and transport of the live tissue sample.

13. The system of claim 12, wherein the tube is sized from about 5 ml to 50 ml in volume.

14. The system of claim 12, wherein the funnel-shaped structure is a thin plastic mesh.

15. The system of claim 12, wherein the structure is solid, formed of plastic, and configured to be separated into two parts and rejoined.

16. The system of claim 15, wherein the two parts are hinged.

17. The system of claim 15, wherein the funnel-shaped structure has an opening at a tapered portion of the funnel-shaped structure configured to allow a buffer to flow or be wicked out while the live tissue sample remains within the funnel-shaped structure.

18. A system for processing a live tissue sample comprising:
at least one filter assembly comprising:
a filter; and
a ring connected to outer portions of the filter, wherein the ring is configured to allow the filter to float on the surface of a fluid.

19. The system of claim 18, wherein the portion of the ring in contact with the filter is hydrophilic, and the opposite portion of the ring is hydrophobic.

20. The system of claim 18, wherein the filter has a pore size from about 2 μm to about 10 μm.

21. The system of claim 18, wherein the ring is formed of biologically inert plastic.

22. The system of claim 18, wherein the ring is formed of a blend of biologically inert waxes.

23. The system of claim 18, wherein the ring is a wrapping film/tape.

24. A system for processing a live tissue sample comprising:
a well-plate insert comprising:
at least one column; and
a semi-permeable filter attached to a bottom of each column that is configured to maintain an air-liquid interface for the live tissue sample after being placed within the column,
wherein after the well-plate insert is placed into a column with liquid at the bottom, the well-plate insert is configured to displace the media without flooding an inner chamber of the well plate where the live tissue sample is located.

25. The system of claim 24, wherein the semi-permeable filter has a pore size from about 2 μm to about 10 μm.

26. The system of claim 24, wherein a microparticle and/or a nutrient within the media travels upwards past the semi-permeable filter without allowing media to permeate into an air chamber within the well plate.
